(19) United States
(12) Patent Application Publication
(10) Pub. No.: US 2021/0405056 A1
(43) Pub. Date: Dec. 30, 2021

(54) METHODS FOR DETECTING MUTANT P53 FUNCTION

(71) Applicant: PMV Pharmaceuticals, Inc., Cranbury, NJ (US)

(72) Inventors: Arnold LEVINE, Doylestown, PA (US); Jonathan OLINER, Garret Park, MD (US); Thomas W. DAVIS, South Orange, NJ (US); Mary Kate McBRAYER, Morganville, NJ (US)

(21) Appl. No.: 17/354,071

(22) Filed: Jun. 22, 2021

Related U.S. Application Data
(60) Provisional application No. 63/043,290, filed on Jun. 24, 2020.

(51) Int. Cl. G01N 33/574

(52) U.S. Cl. CPC G01N 33/57488; G01N 2500/04

(57) ABSTRACT

Mutations in oncogenes and tumor suppressors contribute to the development and progression of cancer. The methods described herein can detect and quantify the ability of a therapeutic agent to reconform a mutant conformation of mutant p53 to a conformation that possesses physiological activity of wild type p53. The disclosed method can be used as a companion diagnostic to detect and quantify the efficacy of a therapeutic agent in reducing the progression of cancers that contain a p53 mutation.

### CROSS-REFERENCE

This application claims the benefit of U.S. Provisional Application No. 63/043,290, filed Jun. 24, 2020, which is incorporated herein by reference.

### BACKGROUND

Cancer, an uncontrolled proliferation of cells, is a multifactorial disease characterized by tumor formation, growth, and in some instances, metastasis. Cells carrying an activated oncogene, damaged genome, or other cancer-promoting alterations can be prevented from replicating through an elaborate tumor suppression network. A central component of this tumor suppression network is p53, one of the most potent tumor suppressors in the cell. Mutant conformations of p53 are implicated in the progression of cancer.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### SUMMARY OF THE INVENTION

In some embodiments, the disclosure provides a method comprising: a) obtaining a sample, wherein the sample comprises an amount of a cell lysate comprising a mutant p53 protein, wherein the mutant p53 protein does not have a mutation at amino acid 175; and a therapeutically-effective amount of a therapeutic agent; i) contacting a first amount of the sample with an antibody specific for a mutant conformation p53 protein in a first contacting area; ii) contacting a second amount of the sample with an antibody specific for a wild type conformation p53 protein in a second contacting area; iii) quantifying an amount of the mutant conformation p53 protein in the first contacting area based on the contacting of the first amount of the sample with the antibody specific for the mutant conformation p53 protein in the first contacting area; iv) quantifying an amount of the wild type conformation p53 protein in the second contacting area based on the contacting of the amount of the second amount of the sample with the antibody specific for the wild type conformation p53 protein in the second contacting area; and b) determining based on a change in the amount of the mutant conformation p53 protein and a change in the amount of the wild type conformation p53 protein from the sample whether the therapeutic candidate reconforms the mutant conformation of the mutant p53 protein into a wild type conformation p53 protein, wherein the wild type conformation p53 protein possesses a biological activity of the wild type p53 protein.

### BRIEF DESCRIPTION OF THE DRAWINGS

FIG. 1 illustrates a sandwich ELISA comprising a primary (capture) antibody, an antigen (Ag), a secondary antibody, a detection antibody, and a substrate.

FIG. 2 shows the concentration-dependent effect of a compound in reducing the % of mutant p53 using an ELISA.

FIG. 3 shows the concentration-dependent effect of a compound in increasing the amount of wild type p53 using an ELISA.

FIG. 4 shows the concentration-dependent effect of a compound on the % of total p53 using an ELISA.

FIG. 5 shows the concentration-dependent effect of a compound (μM) on the amount of mutant p53, wild type p53, and total p53 using NUGC3 cells.

FIG. 6 quantifies the concentration-dependent effect of a compound (μM) on the amount of mutant p53 and wild type p53 using an ELISA.

### DETAILED DESCRIPTION OF THE INVENTION

The present disclosure provides methods for measuring restoration of wild type function to mutant p53. A compound can bind to mutant p53 and restore the ability of the p53 mutant to bind DNA. The restoration of activity of the p53 mutant can allow for the activation of downstream effectors of p53 leading to inhibition of cancer progression.

Cancer is a collection of related diseases characterized by uncontrolled proliferation of cells with the potential to metastasize throughout the body. Cancer can be classified into five broad categories including, for example: carcinomas, which can arise from cells that cover internal and external parts of the body such as the lung, breast, and colon; sarcomas, which can arise from cells that are located in bone, cartilage, fat, connective tissue, muscle, and other supportive tissues; lymphomas, which can arise in the lymph nodes and immune system tissues; leukemia, which can arise in the bone marrow and accumulate in the bloodstream; and adenomas, which can arise in the thyroid, the pituitary gland, the adrenal gland, and other glandular tissues.

Although different cancers can develop in virtually any of the body's tissues, and contain unique features, the basic processes that cause cancer can be similar in all forms of the disease. Cancer begins when a cell breaks free from the normal restraints on cell division and begins to grow and divide out of control. Genetic mutations in the cell can preclude the ability of the cell to repair damaged DNA or initiate apoptosis, and can result in uncontrolled growth and division of cells.

The ability of tumor cell populations to multiply is determined not only by the rate of cell proliferation but also by the rate of cell attrition. Programmed cell death, or apoptosis, represents a major mechanism of cellular attrition. Cancer cells can evade apoptosis through a variety of strategies, for example, through the suppression of p53 function, thereby suppressing expression of pro-apoptotic proteins.

Oncogenes and tumor suppressor genes can regulate the proliferation of cells. Genetic mutations can affect oncogenes and tumor suppressors, potentially activating or suppressing activity abnormally, further facilitating uncontrolled cell division. Whereas oncogenes assist in cellular growth, tumor suppressor genes slow cell division by repairing damaged DNA and activating apoptosis. Cellular oncogenes that can be mutated in cancer include, for example, Cdk1, Cdk2, Cdk3, Cdk4, Cdk6, EGFR, PDGFR, VEGF, HER2, Raf kinase, K-Ras, and myc. Tumor suppressor genes that can be mutated in cancer include, for example, BRCA1, BRCA2, cyclin-dependent kinase inhibitor 1C, Retinoblastoma protein (pRb), PTEN, p16, p27, p53, and p73.

The methods described herein can detect and quantify the ability of a therapeutic agent to reconform a mutant conformation of mutant p53 to a conformation that possesses physiological activity of wild type p53. In some embodiments, the disclosure provides a method comprising: a) obtaining a sample, wherein the sample comprises a therapeutic candidate and a cell lysate, wherein the cell lysate comprises a mutant protein, wherein the mutant protein has a mutant conformation; b) contacting a first amount of the sample with an antibody specific for the mutant protein in a first contacting area; c) contacting a second amount of the sample with an antibody specific for a wild type protein that corresponds to the mutant protein in a second contacting area; d) quantifying an amount of the mutant protein in the first contacting area based on the contacting the first amount of the sample with the antibody specific for the mutant protein in the first contacting area; e) quantifying an amount of the wild type protein in the second contacting area based on the contacting the second amount of the sample with the antibody specific for the wild type protein that corresponds to the mutant protein in the second contacting area; and f) determining based on a change in the amount the mutant protein and a change in the amount of the wild type protein whether the therapeutic candidate reconforms the mutant conformation of the mutant protein into a conformation that possesses a physiological activity of the wild type protein.

The disclosed method can be used as a companion diagnostic to detect and quantify the efficacy of a therapeutic agent in reducing the progression of cancers that contain a p53 mutation.

### Tumor Suppressor p53.

The tumor suppressor protein p53 is a 393 amino acid transcription factor that can regulate cell growth in response to cellular stresses including, for example, UV radiation, hypoxia, oncogene activation, and DNA damage. p53 has various mechanisms for inhibiting the progression of cancer including, for example, initiation of apoptosis, maintenance of genomic stability, cell cycle arrest, induction of senescence, and inhibition of angiogenesis. Due to the critical role of p53 in tumor suppression, p53 is inactivated in almost all cancers either by direct mutation or through perturbation of associated signaling pathways involved in tumor suppression. Homozygous loss of the p53 gene occurs in almost all types of cancer, including carcinomas of the breast, colon, and lung. The presence of certain p53 mutations in several types of human cancer can correlate with less favorable patient prognosis.

In the absence of stress signals, p53 levels are maintained at low levels via the interaction of p53 with Mdm2, an E3 ubiquitin ligase. In an unstressed cell, Mdm2 can target p53 for degradation by the proteasome. Under stress conditions, the interaction between Mdm2 and p53 is disrupted, and p53 accumulates. The critical event leading to the activation of p53 is phosphorylation of the N-terminal domain of p53 by protein kinases, thereby transducing upstream stress signals. The phosphorylation of p53 leads to a conformational change, which can promote DNA binding by p53 and allow transcription of downstream effectors. The activation of p53 can induce, for example, the intrinsic apoptotic pathway, the extrinsic apoptotic pathway, cell cycle arrest, senescence, and DNA repair. p53 can activate proteins involved in the above pathways including, for example, Fas/Apol, KILLER/DR5, Bax, Puma, Noxa, Bid, caspase-3, caspase-6, caspase-7, caspase-8, caspase-9, and p21 (WAF1). Additionally, p53 can repress the transcription of a variety of genes including, for example, c-MYC, Cyclin B, VEGF, RAD51, and hTERT.

Each chain of the p53 tetramer is composed of several functional domains including the transactivation domain (amino acids 1-100), the DNA-binding domain (amino acids 101-306), and the tetramerization domain (amino acids 307-355), which are highly mobile and largely unstructured. Most p53 cancer mutations are located in the DNA-binding core domain of the protein, which contains a central β-sandwich of anti-parallel β-sheets that serves as a basic scaffold for the DNA-binding surface. The DNA-binding surface is composed of two β-turn loops, L2 and L3, which are stabilized by a zinc ion, for example, at Arg175 and Arg248, and a loop-sheet-helix motif. Altogether, these structural elements form an extended DNA-binding surface that is rich in positively-charged amino acids and makes specific contact with various p53 response elements.

Due to the prevalence of p53 mutations in virtually every type of cancer, the reactivation of wild type p53 function in a cancerous cell can be an effective therapy. Mutations in p53 located in the DNA-binding domain of the protein or periphery of the DNA-binding surface result in aberrant protein folding required for DNA recognition and binding. Mutations in p53 can occur, for example, at amino acids Va1143, His168, Arg175, Tyr220, Gly245, Arg248, Arg249, Phe270, Arg273, and Arg282. p53 mutations that can abrogate the activity of p53 include, for example, R175H, Y220C, G245S, R248Q, R248W, R273H, R273C, R282W, and R282H. These p53 mutations can either distort the structure of the DNA-binding site or thermodynamically destabilize the folded protein at body temperature. Wild type function of p53 mutants can be recovered by binding of the p53 mutant to a compound that can shift the folding-unfolding equilibrium towards the folded state, thereby reducing the rate of unfolding and destabilization.

Non-limiting examples of amino acids include: alanine (A, Ala); arginine (R, Arg); asparagine (N, Asn); aspartic acid (D, Asp); cysteine (C, Cys); glutamic acid (E, Glu); glutamine (Q, Gln); glycine (G, Gly); histidine (H, His); isoleucine (I, Ile); leucine (L, Leu); lysine (K, Lys); methionine (M, Met); phenylalanine (F, Phe); proline (P, Pro); serine (S, Ser); threonine (T, Thr); tryptophan (W, Trp); tyrosine (Y, Tyr); and valine (V, Val).

### p53 Reactivating Agents

A therapeutic agent can selectively bind to a p53 mutant and can recover wild type activity of the p53 mutant including, for example, DNA binding function and activation of downstream targets involved in tumor suppression. In some embodiments, a therapeutic agent can reconform the mutant conformation of a mutant protein into a conformation that possess a physiological activity of the corresponding wild type protein. In some embodiments, a therapeutic agent can reconform the mutant conformation of a mutant p53 protein into a conformation that possess a physiological activity of the wild type p53 protein.

In some embodiments, the therapeutic agent of the disclosure is a small molecule. In some embodiments, the therapeutic agent of the disclosure of the disclosure is phenethyl isothiocyanate, CP-31398 dihydrochloride hydrate, APR-246/PRIMA-1^{Met}, PRIMA-1, MIRA-1, PhiKan 083, NSC 319726, RETRA, PK7088, PK7242, or PK5196.

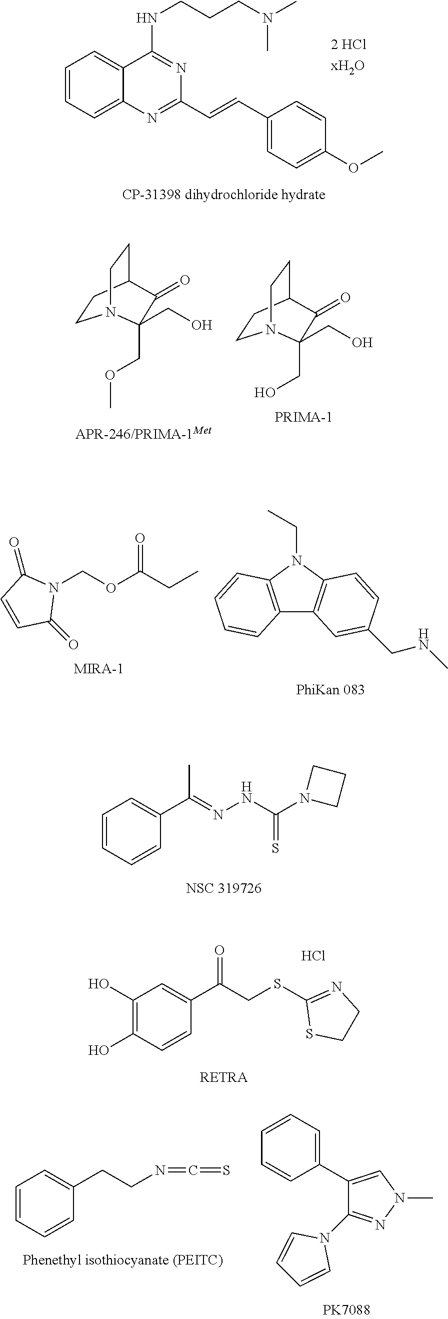

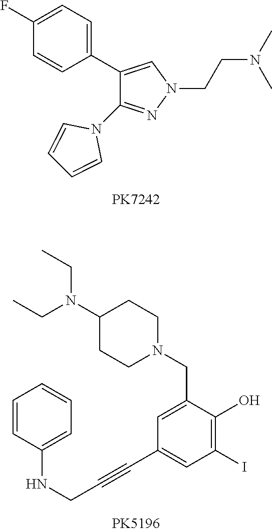

In some embodiments, the therapeutic agent of the disclosure is a peptide. In some embodiments, the therapeutic agent of the disclosure is a peptide of TABLE 1.

TABLE 1

| SEQ ID NO: | Peptide Sequence |
|---|---|
| 1 | EVTFRHSVV |
| 2 | VWVHDSCHANLQNYRNYLP |
| 3 | FWTQSIKERKMLNEHDFER |
| 4 | EHDFEVRGDVVNGRNHQPK |
| 5 | LEVIYMI |
| 6 | LGIDEDEETETAPE |
| 7 | SPLQTPAAPGAAAGPALSPV |
| 8 | LTFEHYWAQLTS |
| 9 | LPNPPERHH |
| 10 | myr-RRLIVRILKLPNPPER |
| 11 | LHSKTLVL |
| 12 | HHPWTHHQRWS |
| 13 | IRILMFLIGCGR |
| 14 | IRILMFLIGCGRRRRRRRRRR |
| 15 | DEDAKFRIRILMRR |
| 16 | LRCLLLLIGRVG |
| 17 | LRCLLLLIGRVGRRRRRRRR |
| 18 | YRRLLIGMMWRRRRRRRRR |
| 19 | YPTQGHLR |
| 20 | IRGRIIR |
| 21 | IRGRIIRRRRRRRRR |
| 22 | myr-RRIRDPRILLLHFD |
| 23 | SFILFIRRGRLG |
| 24 | SFILFIRRGRLGRRRRRRRRR |
| 25 | RRRRRRRGLRGRRIFLIFS |
| 26 | myr-RRKILFIRLMHNKH |
| 27 | HSSHHHPVHSWN |
| 28 | myr-HSSHHHPTVQHRR |
| 29 | HANLHHT |
| 30 | myr-RRKHNKHRPEPDSDER |
| 31 | WNHHHSTPHPAH |
| 32 | WNHHHSTPHPAHRRRRRR |
| 33 | myr-RRHSTPHPD |
| 34 | FPGHTIH |
| 35 | myr-PRVLPSPHTIHPSQYP |

Mechanism of p53-Reactivating Compounds.

Mutant p53-reactivating compounds can selectively bind to a p53 mutant and recover wild type activity of the p53 mutant including, for example, DNA binding function and activation of downstream targets involved in tumor suppression. In some embodiments, a compound or therapeutic agent can selectively bind the mutant protein preferentially over the wild type protein. In some embodiments, a compound or therapeutic agent can selectively bind the mutant p53 protein preferentially over the wild type p53 protein.

In some embodiments, a compound selectively binds to the p53 Y220C mutant. The Y220C mutant is a temperature sensitive mutant, which binds to DNA at lower temperature and is denatured at body temperature. A mutant p53-reactivating compound can stabilize the Y220C mutant to reduce the likelihood of denaturation of the protein at body temperature.

Located in the periphery of the p53 β-sandwich connecting β-strands S7 and S8, the aromatic ring of Y220 is an integral part of the hydrophobic core of the β-sandwich. The Y220C mutation can be highly destabilizing, due to the formation of an internal surface cavity. A mutant p53-reactivating compound can bind to and occupy this surface crevice to stabilize the β-sandwich, thereby restoring wild type p53 DNA-binding activity.

In some embodiments, a compound selectively binds to the p53 Y220S mutant. In some embodiments, a compound selectively binds to the p53 R273H mutant. In some embodiments, a compound selectively binds to the p53 R273C mutant. In some embodiments, a compound selectively binds to the p53 R282W mutant.

To determine the ability of a mutant p53-reactivating compound to bind and stabilize mutant p53, assays can be employed to detect, for example, a conformational change in the p53 mutant or activation of wild type p53 targets. Conformational changes in p53 can be measured by, for example, an enzyme-linked immunosorbent assay (ELISA). In some embodiments, conformational changes in p53 can also be measured by differential scanning fluorimetry (DSF), isothermal titration calorimetry (ITC), nuclear magnetic resonance spectrometry (NMR), or X-ray crystallography. Additionally, antibodies specific for the wild type of mutant conformation of p53 can be used to detect a conformational change via, for example, immunoprecipitation (IP), immunofluorescence (IF), or immunoblotting.

Methods used to detect the ability of the p53 mutant to bind DNA can include, for example, DNA affinity immunoblotting, modified ELISA, electrophoretic mobility shift assay (EMSA), fluorescence resonance energy transfer (FRET), homogeneous time-resolved fluorescence (HTRF), and a chromatin immunoprecipitation (ChIP) assay.

To determine whether a mutant p53-reactivating compound is able to reactivate the transcriptional activity of p53, the activation of downstream targets in the p53 signaling cascade can be measured. Activation of p53 effector proteins can be detected by, for example, immunohistochemistry (IHC-P), reverse transcription polymerase chain reaction (RT-PCR), and western blotting. The activation of p53 can also be measured by the induction of apoptosis via the caspase cascade and using methods including, for example, Annexin V staining, TUNEL assays, pro-caspase and caspase levels, and cytochrome c levels. Another consequence of p53 activation is senescence, which can be measured using methods such as β-galactosidase staining.

A p53 mutant that can be used to determine the effectiveness of a mutant p53-reactivating compound to increase the DNA binding ability of a p53 mutant is a p53 truncation mutant, which contains only amino acids 94-312, encompassing the DNA-binding domain of p53. For example, the sequence of the p53 Y220C mutant used for testing compound efficacy can be

(SEQ ID NO: 36)
SSSVPSQ KTYQGSYGFR LGFLHSGTAK SVTCTYSPAL
NKMFCQLAKT CPVQLWVDST PPPGTRVRAM AIYKQSQHMT
EVVRRCPHHE RCSDSDGLAP PQHLIRVEGN LRVEYLDDRN
TFRHSVVVPC EPPEVGSDCT TIHYNYMCNS SCMGGMNRRP
ILTIITLEDS SGNLLGRNSF EVHVCACPGR DRRTEEENLR
KKGEPHHELP PGSTKRALSN NT

A mutant p53-reactivating compound can increase the ability of a p53 mutant to bind DNA by at least or up to about 0.1%, at least or up to about 0.2%, at least or up to about 0.3%, at least or up to about 0.4%, at least or up to about 0.5%, at least or up to about 0.6%, at least or up to about 0.7%, at least or up to about 0.8%, at least or up to about 0.9%, at least or up to about 1%, at least or up to about 2%, at least or up to about 3%, at least or up to about 4%, at least or up to about 5%, at least or up to about 6%, at least or up to about 7%, at least or up to about 8%, at least or up to about 9%, at least or up to about 10%, at least or up to about 11%, at least or up to about 12%, at least or up to about 13%, at least or up to about 14%, at least or up to about 15%, at least or up to about 16%, at least or up to about 17%, at least or up to about 18%, at least or up to about 19%, at least or up to about 20%, at least or up to about 21%, at least or up to about 22%, at least or up to about 23%, at least or up to about 24%, at least or up to about 25%, at least or up to about 26%, at least or up to about 27%, at least or up to about 28%, at least or up to about 29%, at least or up to about 30%, at least or up to about 31%, at least or up to about 32%, at least or up to about 33%, at least or up to about 34%, at least or up to about 35%, at least or up to about 36%, at least or up to about 37%, at least or up to about 38%, at least or up to about 39%, at least or up to about 40%, at least or up to about 41%, at least or up to about 42%, at least or up to about 43%, at least or up to about 44%, at least or up to about 45%, at least or up to about 46%, at least or up to about 47%, at least or up to about 48%, at least or up to about 49%, at least or up to about 50%, at least or up to about 51%, at least or up to about 52%, at least or up to about 53%, at least or up to about 54%, at least or up to about 55%, at least or up to about 56%, at least or up to about 57%, at least or up to about 58%, at least or up to about 59%, at least or up to about 60%, at least or up to about 61%, at least or up to about 62%, at least or up to about 63%, at least or up to about 64%, at least or up to about 65%, at least or up to about 66%, at least or up to about 67%, at least or up to about 68%, at least or up to about 69%, at least or up to about 70%, at least or up to about 71%, at least or up to about 72%, at least or up to about 73%, at least or up to about 74%, at least or up to about 75%, at least or up to about 76%, at least or up to about 77%, at least or up to about 78%, at least or up to about 79%, at least or up to about 80%, at least or up to about 81%, at least or up to about 82%, at least or up to about 83%, at least or up to about 84%, at least or up to about 85%, at least or up to about 86%, at least or up to about 87%, at least or up to about 88%, at least or up to about 89%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, at least or up to about 100%, at least or up to about 125%, at least or up to about 150%, at least or up to about 175%, at least or up to about 200%, at least or up to about 225%, or at least or up to about 250% as compared to the ability of the p53 mutant to bind DNA in the absence of a mutant p53-reactivating compound.

A mutant p53-reactivating compound can increase the activity of the p53 mutant that is, for example, at least or up to about 2-fold, at least or up to about 3-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 11-fold, at least or up to about 12-fold, at least or up to about 13-fold, at least or up to about 14-fold, at least or up to about 15-fold, at least or up to about 16-fold, at least or up to about 17-fold, at least or up to about 18-fold, at least or up to about 19-fold, at least or up to about 20-fold, at least or up to about 25-fold, at least or up to about 30-fold, at least or up to about 35-fold, at least or up to about 40-fold, at least or up to about 45-fold, at least or up to about 50-fold, at least or up to about 55-fold, at least or up to about 60-fold, at least or up to about 65-fold, at least or up to about 70-fold, at least or up to about 75-fold, at least or up to about 80-fold, at least or up to about 85-fold, at least or up to about 90-fold, at least or up to about 95-fold, at least or up to about 100-fold, at least or up to about 110-fold, at least or up to about 120-fold, at least or up to about 130-fold, at least or up to about 140-fold, at least or up to about 150-fold, at least or up to about 160-fold, at least or up to about 170-fold, at least or up to about 180-fold, at least or up to about 190-fold, at least or up to about 200-fold, at least or up to about 250-fold, at least or up to about 300-fold, at least or up to about 350-fold, at least or up to about 400-fold, at least or up to about 450-fold, at least or up to about 500-fold, at least or up to about 550-fold, at least or up to about 600-fold, at least or up to about 650-fold, at least or up to about 700-fold, at least or up to about 750-fold, at least or up to about 800-fold, at least or up to about 850-fold, at least or up to about 900-fold, at least or up to about 950-fold, at least or up to about 1,000-fold, at least or up to about 1,500-fold, at least or up to about 2,000-fold, at least or up to about 3,000-fold, at least or up to about 4,000-fold, at least or up to about 5,000-fold, at least or up to about 6,000-fold, at least or up to about 7,000-fold, at least or up to about 8,000-fold, at least or up to about 9,000-fold, or at least or up to about 10,000-fold greater than the activity of the p53 mutant in the absence of the compound.

A mutant p53-reactivating compound can be used, for example, to induce apoptosis, cell cycle arrest, or senescence in a cell. In some embodiments, the cell is a cancer cell. In some embodiments, the cell carries a mutation in p53.

### Enzyme-Linked Immunosorbent Assay (ELISA)

An enzyme-linked immunosorbent assay (ELISA) is a plate-based assay technique designed for detecting and quantifying substances, such as peptides, proteins, and antibodies. Detection is accomplished by assessing the conjugated enzyme activity via incubation with a substrate to produce a measurable product. The most crucial element of the detection strategy is a highly specific antibody-antigen interaction. The present disclosure provides an ELISA to detect the ability of a compound or agent to re-activate mutant p53 to wild type p53. In some embodiments, the disclosure provides an ELISA to detect the ability of a compound to re-activate mutant p53 to wild type p53.

An ELISA of the disclosure can be performed in a multi-well polystyrene plate, which can passively bind an antibody or protein. In some embodiments, an ELISA of the disclosure can be performed in a 96-well polystyrene plate. In some embodiments, an ELISA of the disclosure can be performed in a 384-well polystyrene plate.

In a direct ELISA, an antigen is immobilized in the well of an ELISA plate, and the antigen is then detected by an antibody directly conjugated to an enzyme, such as HRP. In some embodiments, a direct ELISA is used to detect the ability of a compound to change the conformation of mutant p53 to wild type p53.

In an indirect ELISA, an antigen is absorbed to a well in an ELISA plate. An unlabeled primary antibody binds to the specific antigen, and an enzyme-linked conjugated secondary antibody that is directed against the primary antibody is applied. In some embodiments, an indirect ELISA is used to detect the ability of a compound to change the conformation of mutant p53 to wild type p53.

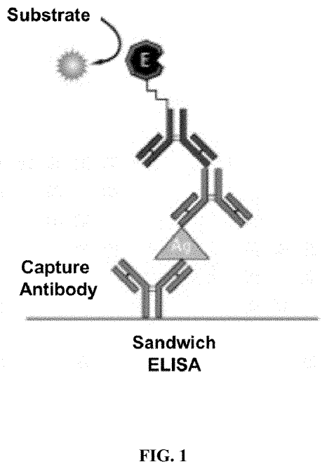

In a sandwich ELISA, a well of an ELISA plate is coated with a capture antibody. The compound is added, followed by a detection antibody. In some embodiments, a sandwich ELISA is used to detect the ability of a compound to change the conformation of mutant p53 to wild type p53. In some embodiments, the detection antibody can be enzyme conjugated (direct sandwich ELISA). In some embodiments, the detection antibody can be unlabeled, and a secondary enzyme-conjugated antibody can be used (indirect sandwich ELISA). FIG. 1 illustrates a sandwich ELISA comprising a primary (capture) antibody, an antigen (Ag), a secondary antibody, a detection antibody, and a substrate.

In a competitive ELISA, a compound is used to coat a multi-well plate. Following standard blocking and washing steps, samples containing an unknown antigen are added. A labeled detection antibody is then applied for detection using relevant substrates. In some embodiments, a competition ELISA is used to detect the ability of a compound to change the conformation of mutant p53 to wild type p53.

A detection enzyme of the disclosure can be linked directly to a primary antibody or introduced through a secondary antibody that recognizes the primary antibody. In some embodiments, a detection enzyme of the disclosure can be linked directly to a primary antibody. In some embodiments, a detection enzyme of the disclosure can be introduced through a secondary antibody that recognizes the primary antibody. In some embodiments, the detection enzyme is linked to streptavidin and the primary antibody is biotin labeled. In some embodiments, the detection enzyme is horseradish peroxidase (HRP). In some embodiments, the detection enzyme is alkaline phosphatase (AP).

In some embodiments, an antigen can be directly immobilized on a surface by absorbing the antigen to the assay plate. In some embodiments, an antigen can be indirectly immobilized on a plate using a capture antibody that has been attached to the surface of a plate.

In some embodiments, a method of the disclosure comprises a) obtaining a sample, wherein the sample comprises a therapeutic candidate and a cell lysate, wherein the cell lysate comprises a mutant protein, wherein the mutant protein has a mutant conformation; b) contacting a first amount of the sample with an antibody specific for the mutant protein in a first contacting area; c) contacting a second amount of the sample with an antibody specific for a wild type protein that corresponds to the mutant protein in a second contacting area; d) quantifying an amount of the mutant protein in the first contacting area based on the contacting the first amount of the sample with the antibody specific for the mutant protein in the first contacting area; e) quantifying an amount of the wild type protein in the second contacting area based on the contacting the second amount of the sample with the antibody specific for the wild type protein that corresponds to the mutant protein in the second contacting area; and f) determining based on a change in the amount the mutant protein and a change in the amount of the wild type protein whether the therapeutic candidate reconforms the mutant conformation of the mutant protein into a conformation that possesses a physiological activity of the wild type protein.

In some embodiments, the first contacting area is a first well with the antibody specific for the mutant protein immobilized to a surface within the first well, and the second contacting area is a second well with the antibody specific for the wild type protein immobilized to a surface within the second well.

In some embodiments, the obtaining the sample comprises preparing the sample by contacting the therapeutic candidate with the cell lysate. In some embodiments, the cell lysate is obtained from NUGC-3 cells. In some embodiments, the cell lysate is obtained from a tumor. In some embodiments, the cell lysate is obtained from a breast cancer tumor. In some embodiments, the cell lysate is obtained from a prostate cancer tumor. In some embodiments, the cell lysate is obtained from a lung cancer tumor. In some embodiments, the cell lysate is obtained from a skin cancer tumor.

An ELISA of the disclosure can use a colorimetric substrate to detect or quantify the ability of a compound to change the conformation of mutant p53 to wild type p53. In some embodiments, p-nitrophenyl phosphate disodium salt (PNPP) is used as a colorimetric substrate to detect AP. In some embodiments, 2,2′-azinobis-[3-ethylbenzothiazoline-6-sulfonic acid]-diammonium salt (ABTS) is used to detect HRP. In some embodiments, o-phenylenediamine dihydrochloride (OPD) is used to detect HRP. In some embodiments, 3,3′,5,5′-tetramethylbenzidine (TMB) is used to detect HRP.

In some embodiments, the quantifying comprises visualizing the amount of the wild type protein and the amount of the mutant protein. In some embodiments, the visualizing comprises contacting the wild type protein and the mutant protein with a detection antibody. In some embodiments, the detection antibody is a biotinylated antibody. In some embodiments, the visualizing further comprises contacting the detection antibody with an enzyme. In some embodiments, the enzyme is HRP. In some embodiments, the enzyme is HRP-conjugated streptavidin.

An ELISA of the disclosure can use a chemiluminescent substrate to detect or quantify the ability of a compound to change the conformation of mutant p53 to wild type p53. In some embodiments, CSPD®, CDP-Star™ substrates can be used to detect AP. In some embodiments, a formulation comprising 1,2-dioxetane chemiluminescent substrate, such as DynaLight™ substrate with RapidGlow™ Enhancer, can be used to detect AP. In some embodiments, SuperSignal™ ELISA Pico Chemiluminescent substrate can be used to detect HRP. In some embodiments, SuperSignal™ ELISA Femto Maximum Sensitivity Substrate can be used to detect HRP.

An ELISA of the disclosure can use a substrate that produces a fluorescent substrate product. A substrate product of the disclosure can be detected using a spectrophotometer, fluorometer, or a luminometer. In some embodiments, a substrate product of PNPP is detected at 405 nm using a spectrophotometer. In some embodiments, a substrate product of ABTS is detected at 410 nm or 650 nm using a spectrophotometer. In some embodiments, a substrate product of OPD is detected at 492 nm using a spectrophotometer. In some embodiments, a substrate product of TMB is detected at 450 nm, 650 nm, or 652 nm using a spectrophotometer. In some embodiments, the substrate product of TMB is 3,3′,5,5′-tetramethylbenzidine diamine and is detected at 450 nm. In some embodiments, a substrate product of SuperSignal™ ELISA Pico is detected at 425 nm using a luminometer. In some embodiments, a substrate product of SuperSignal™ ELISA Femto is detected at 425 nm using a luminometer.

### Methods of Use

In some embodiments, an ELISA can be used to detect or quantify the ability of a mutant p53-reactivating compound to reconform a mutant conformation of mutant p53 into a conformation that possesses a physiological activity of the wild type protein. In some embodiments, an ELISA can be used to detect or quantify the ability of a mutant Y220 p53-reactivating compound to reconform a mutant conformation of a mutant p53 into a conformation that possess a physiological activity of the wild type protein. In some embodiments, the mutant Y220 p53 is Y220C p53. In some embodiments, the mutant Y220 p53 is Y220S p53. In some embodiments, an ELISA can be used to detect or quantify the ability of a mutant R273 p53-reactivating compound to reconform a mutant conformation of a mutant p53 into a conformation that possess a physiological activity of the wild type protein. In some embodiments, the mutant R273 p53 is R273H p53. In some embodiments, the mutant R273 p53 is R273C p53. In some embodiments, an ELISA can be used to detect or quantify the ability of a mutant R282 p53-reactivating compound to reconform a mutant conformation of a mutant p53 into a conformation that possess a physiological activity of the wild type protein. In some embodiments, the mutant R282 p53 is R282W p53.

In some embodiments, a p53-reactivating compound can slow the proliferation of cancer cell lines, or kill cancer cells. Non-limiting examples of cancer that contain a p53 mutation, wherein a p53-reactivating compound can reconform a mutant p53 into a conformation that possesses a physiological activity of the wild type protein include: acute lymphoblastic leukemia, acute myeloid leukemia, adrenocortical carcinoma, AIDS-related cancers, AIDS-related lymphoma, anal cancer, appendix cancer, astrocytomas, basal cell carcinoma, bile duct cancer, bladder cancer, bone cancers, brain tumors, such as cerebellar astrocytoma, cerebral astrocytoma/malignant glioma, ependymoma, medulloblastoma, supratentorial primitive neuroectodermal tumors, visual pathway and hypothalamic glioma, breast cancer, bronchial adenomas, Burkitt lymphoma, carcinoma of unknown primary origin, central nervous system lymphoma, cerebellar astrocytoma, cervical cancer, childhood cancers, chronic lymphocytic leukemia, chronic myelogenous leukemia, chronic myeloproliferative disorders, colon cancer, cutaneous T-cell lymphoma, desmoplastic small round cell tumor, endometrial cancer, ependymoma, esophageal cancer, Ewing's sarcoma, germ cell tumors, gallbladder cancer, gastric cancer, gastrointestinal carcinoid tumor, gastrointestinal stromal tumor, gliomas, hairy cell leukemia, head and neck cancer, heart cancer, hepatocellular (liver) cancer, Hodgkin lymphoma, Hypopharyngeal cancer, intraocular melanoma, islet cell carcinoma, Kaposi sarcoma, kidney cancer, laryngeal cancer, lip and oral cavity cancer, liposarcoma, liver cancer, lung cancers, such as non-small cell and small cell lung cancer, lymphomas, leukemias, macroglobulinemia, malignant fibrous histiocytoma of bone/osteosarcoma, medulloblastoma, melanomas, mesothelioma, metastatic squamous neck cancer with occult primary, mouth cancer, multiple endocrine neoplasia syndrome, myelodysplastic syndromes, myeloid leukemia, nasal cavity and paranasal sinus cancer, nasopharyngeal carcinoma, neuroblastoma, non-Hodgkin lymphoma, non-small cell lung cancer, oral cancer, oropharyngeal cancer, osteosarcoma/malignant fibrous histiocytoma of bone, ovarian cancer, ovarian epithelial cancer, ovarian germ cell tumor, pancreatic cancer, pancreatic cancer islet cell, paranasal sinus and nasal cavity cancer, parathyroid cancer, penile cancer, pharyngeal cancer, pheochromocytoma, pineal astrocytoma, pineal germinoma, pituitary adenoma, pleuropulmonary blastoma, plasma cell neoplasia, primary central nervous system lymphoma, prostate cancer, rectal cancer, renal cell carcinoma, renal pelvis and ureter transitional cell cancer, retinoblastoma, rhabdomyosarcoma, salivary gland cancer, sarcomas, skin cancers, skin carcinoma merkel cell, small intestine cancer, soft tissue sarcoma, squamous cell carcinoma, stomach cancer, T-cell lymphoma, throat cancer, thymoma, thymic carcinoma, thyroid cancer, trophoblastic tumor (gestational), cancers of unknown primary site, urethral cancer, uterine sarcoma, vaginal cancer, vulvar cancer, Waldenström macroglobulinemia, and Wilms tumor.

### EXAMPLES

### Example 1

### Cell Harvesting

NUGC3 Cell media was aspirated, and plates were washed twice with cold phosphate buffered saline (PBS). Lysis buffer was added to the plates, and the plates were left on ice for 5 to 10 minutes. For 6-well plates, 125 μL to 150 μL lysis buffer was added to each well. For 10 cm dishes, 300 μL to 500 μL lysis buffer was added to each dish. The plates were optionally shaken at 4° C. for 5 to 10 minutes. The cells were scraped from the plates using a cell scraper, and the resulting lysate was added to an Eppendorf Tube®, which was spun by a micro-centrifuge at maximum speed for 15 minutes. The resulting supernatant was transferred into a clean Eppendorf Tube® and used in the protein assay.

EXAMPLE 2: Bicinchoninic acid (BCA) Protein Assay

Standards were added in duplicate in rows 1 and 2 of a plate with volumes of 2000 μg/mL, 150 μg/mL, 1000 μg/mL, 750 μg/mL, 500 μg/mL, 250 μg/mL, 125 μg/mL. Samples were added in duplicate in rows 3-12 with the different volumes. 2 μL of lysis buffer and 10 μL of the standard were added to the standard wells. In row H, 1 μL of lysis buffer and 10 μL of PBS were added. For the wells containing ‘unknown’ samples, 2 μL of ‘unknown’ and 10 μL of PBS were added to each well. 4 blank wells were prepared with 1 μL of lysis buffer and 10 μL of PBS.

A Pierce™ BCA Protein Assay Kit was used. A 1:50 dilution of reagents was prepared by mixing 1 mL of reagent B with 49 mL of reagent A. The mixture of reagent A and reagent B was incubated at 37° C. for 30 minutes. The results of the protein assay were analyzed using SoftMax® Pro. The plates were shaken for 10 seconds, and absorbance of the plates was read at 562 nm using a spectrophotometer.

Day 1: Wild type p53 (PAb1620); mutant p53 (PAb240), and total p53 (PAb1801) antibodies were used for the enzyme-linked immunosorbent assay ELISA. The wild type p53 antibody had an initial concentration of 0.5 mg/mL. 30 μL of the 0.5 mg/mL wild type p53 antibody solution was diluted in 10 mL of lx PBS to achieve a final wild-type p53 antibody concentration of 1.5 μg/mL. The mutant p53 antibody had an initial concentration of 1 mg/mL. 10 μL of the 1 mg/mL mutant p53 antibody solution was diluted in 10 mL of 1X PBS to achieve a final mutant p53 antibody concentration of 1 μg/mL. The total p53 antibody had an initial concentration of 1 mg/mL. 3.13 μL of the 1 mg/mL total p53 antibody solution was diluted in 10 mL of 1X PBS to obtain a final total p53 antibody concentration of 0.313 ng/mL. One plate was used per antibody. The plates were coated with 100 μL of antibody per well and incubated overnight at 4° C. without shaking.

Day 2: While the plates were blocking, the samples were diluted. The ELISA plate was washed with 200 μL of PBS with 0.05% tween-20 (wash buffer). The plate was blocked for one hour at room temperature on a shaker in PBS with 0.05% tween-20 and 1% BSA (blocking buffer). The plate was then washed with 200 μL of PBS with 0.05% tween-20 (wash buffer).

All samples were diluted to the same concentration in PBS containing 0.05% tween-20 and 1% BSA (blocking buffer) based on the BCA protein assay. A multichannel pipette was used to transfer samples in small tubes laid out in a 96-well format. The samples were added to the ELISA plate. 100 μL of the wild type p53 antibody (0.06 mg/mL), mutant p53 antibody (0.03 mg/mL), and total p53 antibody (0.015 mg/mL) were added to each well on the plate.

Day 3: The plate was washed three times with the wash buffer using 200 μL/mL of the wash buffer. The plates were incubated with 100 μL/well of rabbit biotinylated p53 antibody (detection antibody) for 1 hour at room temperature. The protein concentration of the detection antibody was diluted to 0.025 mg/mL by adding 2.5 μL of a 0.1 mg/mL stock solution into 10 mL of buffer. The plate was washed three times with 200 μL/well of wash buffer.

The plates were incubated with horseradish peroxidase (HRP)-streptavidin for 30 minutes. The HRP-streptavidin was diluted to 1:10,000 in the blocking buffer by adding 1.25 μL of the stock solution to 12.5 mL of the blocking buffer. The plates were covered with foil or paper during incubation. The plates were then washed three times with the wash buffer using 200 μL/well.

The plate was developed by adding 100 μL of 3,3′,5,5′-tetramethylbenzidine (TMB) until a blue color was observed. The plate treated with wild type p53 antibody was developed for approximately 5 minutes; the plate treated with mutant p53 antibody was developed for approximately 3 minutes; and the plate treated with total p53 antibody was developed for 2-5 minutes. 200 μL of a 0.16M sulfuric acid solution was added to stop the reaction. The plates were read the plate reader at 450 nm.

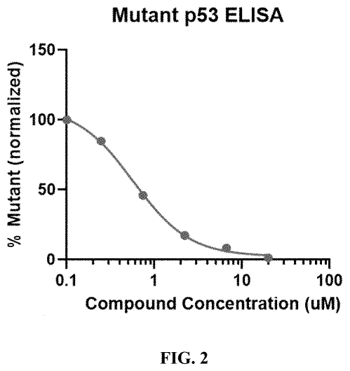

FIG. 2 shows a decrease in the % of mutant p53 of a sample with increasing concentrations of a compound of the disclosure.

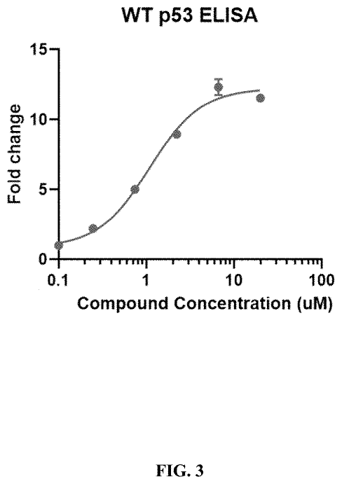

FIG. 3 shows an increase in the amount of wild type p53 conformation with increasing concentrations of a compound of the disclosure.

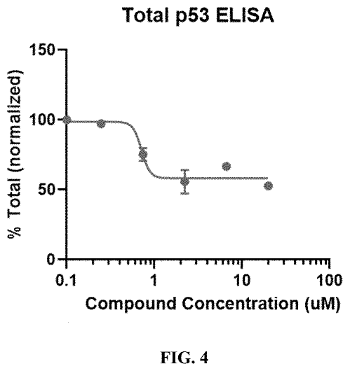

FIG. 4 shows a change in the % of total p53 detected by ELISA upon treatment with a compound of the disclosure.

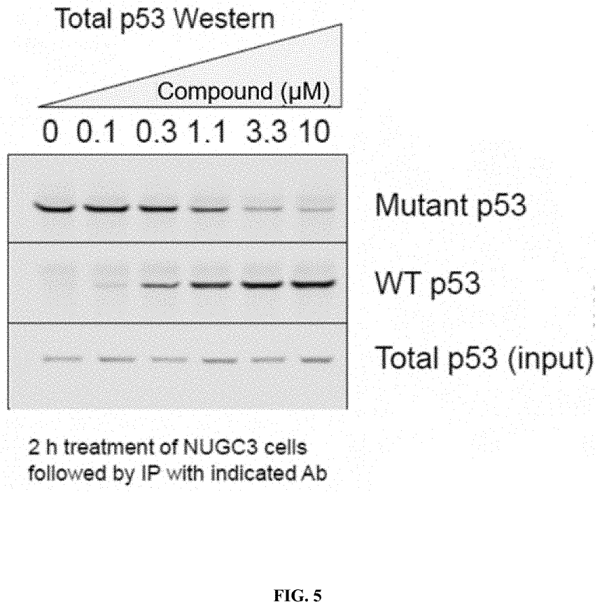

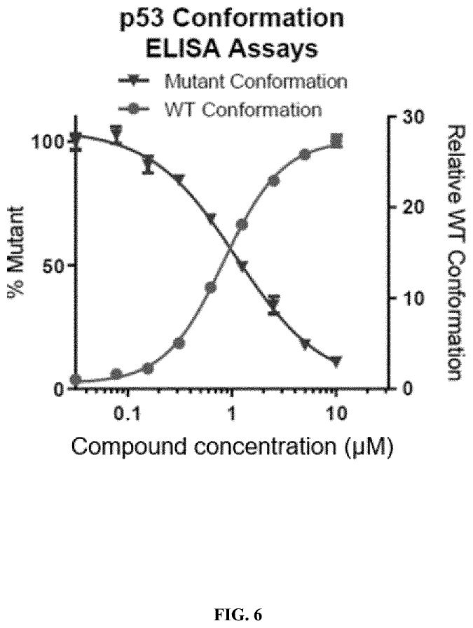

FIG. 5 compares the amounts of mutant p53, wild type p53, and total p53 detected upon treatment of NUGC3 cells with increasing amounts of a compound of the disclosure. FIG. 6 quantifies the amounts of mutant p53, wild type p53, and total p53 detected in FIG. 5. Quantification of mutant and wild type p53 ELISAS of FIG. 5 and FIG. 6 are shown in TABLE 2.

TABLE 2

| | | | Mutant p53 | | WT fold |
|---|---|---|---|---|---|
| | | Mutant p53 | values | WT p53 | change |
| | | Raw values | normalized | Raw values | relative |
| | | (O.D) | (% DMSO) | (O.D.) | to DMSO |
| 10 | μM | 0.077 | 10.748 | 1.418 | 27.312 |
| 5 | μM | 0.127 | 17.775 | 1.342 | 25.840 |
| 2.5 | μM | 0.242 | 33.959 | 1.190 | 22.925 |
| 1.25 | μM | 0.352 | 49.345 | 0.941 | 18.118 |
| 0.625 | μM | 0.489 | 68.500 | 0.581 | 11.182 |
| 0.3125 | μM | 0.602 | 84.348 | 0.262 | 5.049 |
| 0.15625 | μM | 0.647 | 90.647 | 0.116 | 2.235 |
| 0.078125 | μM | 0.732 | 102.634 | 0.084 | 1.621 |

| | | | | |
|---|---|---|---|---|
| DMSO | 0.714 | 100 | 0.052 | 1.000 |

### EMBODIMENTS

The following non-limiting embodiments provide illustrative examples of the invention, but do not limit the scope of the invention.

Embodiment 1. A method comprising: a) obtaining a sample, wherein the sample comprises an amount of a cell lysate comprising a mutant p53 protein, wherein the mutant p53 protein does not have a mutation at amino acid 175; and a therapeutically-effective amount of a therapeutic agent; i) contacting a first amount of the sample with an antibody specific for a mutant conformation p53 protein in a first contacting area; ii) contacting a second amount of the sample with an antibody specific for a wild type conformation p53 protein in a second contacting area; iii) quantifying an amount of the mutant conformation p53 protein in the first contacting area based on the contacting of the first amount of the sample with the antibody specific for the mutant conformation p53 protein in the first contacting area; iv) quantifying an amount of the wild type conformation p53 protein in the second contacting area based on the contacting of the amount of the second amount of the sample with the antibody specific for the wild type conformation p53 protein in the second contacting area; and b) determining based on a change in the amount of the mutant conformation p53 protein and a change in the amount of the wild type conformation p53 protein from the sample whether the therapeutic candidate reconforms the mutant conformation of the mutant p53 protein into a wild type conformation p53 protein, wherein the wild type conformation p53 protein possesses a biological activity of the wild type p53 protein.

Embodiment 2. The method of embodiment 1, further comprising: before the determining, obtaining a second sample, wherein the second sample comprises a second amount of the cell lysate; i) contacting a first amount of the second sample with the antibody specific for the mutant conformation p53 protein in a third contacting area; ii) contacting a second amount of the second sample with the antibody specific for the wild type conformation p53 protein in a fourth contacting area; iii) quantifying an amount of the mutant conformation p53 protein in the third contacting area based on the contacting of the first amount of the second sample with the antibody specific for the mutant conformation p53 protein in the third contacting area; iv) quantifying an amount of the wild type conformation p53 protein in the fourth contacting area based on the contacting of the second amount of the second sample with the antibody specific for the wild type conformation p53 protein in the fourth contacting area.

Embodiment 3. The method of embodiment 1 or 2, wherein the mutant p53 protein has a mutation at amino acid 220.

Embodiment 4. The method of any one of embodiments 1-3, wherein the mutant p53 protein is Y220C mutant p53.

Embodiment 5. The method of any one of embodiments 1-3, wherein the mutant p53 protein is Y220S mutant p53.

Embodiment 6. The method of any one of embodiments 1-5, wherein the mutant p53 protein has a mutation at amino acid 273.

Embodiment 7. The method of any one of embodiments 1-6, wherein the mutant p53 protein is R273C mutant p53.

Embodiment 8. The method of any one of embodiments 1-6, wherein the mutant p53 protein is R273H mutant p53

Embodiment 9. The method of any one of embodiments 1-8, wherein the obtaining the sample comprises preparing the sample by contacting the therapeutic candidate with the cell lysate.

Embodiment 10. The method of any one of embodiments 1-9, wherein the therapeutic candidate is a small molecule.

Embodiment 11. The method of any one of embodiments 1-9, wherein the therapeutic candidate is a peptide.

Embodiment 12. The method of any one of embodiments 1-11, wherein the therapeutic candidate selectively binds the mutant p53 protein preferentially over the wild type p53 protein.

Embodiment 13. The method of any one of embodiments 1-12, wherein the first contacting area is a first well with the antibody specific for the mutant conformation p53 protein immobilized to a surface within the first well, and the second contacting area is a second well with the antibody specific for the wild type conformation p53 protein immobilized to a surface within the second well.

Embodiment 14. The method of any one of embodiments 1-13, wherein the quantifying the amount of the mutant conformation p53 protein comprises: a) contacting the first amount of the sample with the antibody specific for the mutant conformation p53 protein, wherein the mutant conformation p53 and the antibody specific for the mutant conformation p53 form a first complex; b) contacting the first complex with a detection antibody, wherein the first complex and the detection antibody form a second complex; c) contacting the second complex with an enzyme, wherein the second complex and the enzyme form a third complex; d) contacting the third complex with a substrate, wherein the contacting of the third complex with the substrate produces an oxidation product of the substrate; and e) visualizing the oxidation product by spectrophotometry.

Embodiment 15. The method of any one of embodiments 1-14, wherein the quantifying the amount of the wild type conformation p53 protein comprises: a) contacting the first amount of the sample with the antibody specific for the wild type conformation p53 protein, wherein the wild type conformation p53 and the antibody specific for the wild type conformation p53 form a first complex; b) contacting the first complex with a detection antibody, wherein the first complex and the detection antibody form a second complex; c) contacting the second complex with an enzyme, wherein the second complex and the enzyme form a third complex; d) contacting the third complex with a substrate, wherein the contacting of the third complex with the substrate produces an oxidation product of the substrate; and e) visualizing the oxidation product by spectrophotometry.

Embodiment 16. The method of any one of embodiments 1-15, wherein the quantifying the amount of the mutant conformation p53 protein comprises visualizing the amount of the mutant conformation p53 protein.

Embodiment 17. The method of any one of embodiments 1-16, wherein the quantifying the amount of the wild type conformation p53 protein comprises visualizing the amount of the wild type p53 conformation protein.

Embodiment 18. The method of any one of embodiments 1-16, wherein the visualizing comprises contacting the mutant conformation p53 protein with a detection antibody.

Embodiment 19. The method of any one of embodiments 1-17, wherein the visualizing comprises contacting the wild type conformation p53 protein with a detection antibody.

Embodiment 20. The method of embodiments 18 or 19, wherein the detection antibody is a biotinylated antibody.

Embodiment 21. The method of embodiments 18 or 19, wherein the visualizing further comprises contacting the detection antibody with an enzyme.

Embodiment 22. The method of embodiment 21, wherein the enzyme is horseradish peroxidase (HRP).

Embodiment 23. The method of embodiment 21, wherein the enzyme is HRP-conjugated streptavidin.

Embodiment 24. The method of embodiment 21, wherein the visualizing further comprises contacting the enzyme with a substrate.

Embodiment 25. The method of embodiment 24, wherein the substrate is 3,3′,5,5′-tetramethylbenzidine (TMB).

Embodiment 26. The method of embodiment 25, further comprising visualizing an oxidation product of TMB by spectrophotometry.

Embodiment 27. The method of embodiment 26, wherein the oxidation product is 3,3′,5,5′-tetramethylbenzidine diimine.

Embodiment 28. The method of embodiment 27, wherein the 3,3′,5,5′-tetramethylbenzidine diimine is visualized at a wavelength of 450 nm.

Embodiment 29. The method of any one of embodiments 1-28, wherein the cell lysate is obtained from NUGC-3 cells.

Embodiment 30. The method of any one of embodiments 1-29, wherein the cell lysate is obtained from a tumor.

Embodiment 31. The method of embodiment 30, wherein the tumor is a breast cancer tumor.

Embodiment 32. The method of embodiment 30 wherein the tumor is a prostate cancer tumor.

Embodiment 33. The method of embodiment 30, wherein the tumor is a lung cancer tumor.

Embodiment 34. The method of embodiment 30, wherein the tumor is a skin cancer tumor.

1. A method comprising:
a) obtaining a sample, wherein the sample comprises an amount of a cell lysate comprising a mutant p53 protein, wherein the mutant p53 protein does not have a mutation at amino acid 175; and a therapeutically-effective amount of a therapeutic agent;
i) contacting a first amount of the sample with an antibody specific for a mutant conformation p53 protein in a first contacting area;
ii) contacting a second amount of the sample with an antibody specific for a wild type conformation p53 protein in a second contacting area;
iii) quantifying an amount of the mutant conformation p53 protein in the first contacting area based on the contacting of the first amount of the sample with the antibody specific for the mutant conformation p53 protein in the first contacting area;
iv) quantifying an amount of the wild type conformation p53 protein in the second contacting area based on the contacting of the amount of the second amount of the sample with the antibody specific for the wild type conformation p53 protein in the second contacting area; and
b) determining based on a change in the amount of the mutant conformation p53 protein and a change in the amount of the wild type conformation p53 protein from the sample whether the therapeutic candidate reconforms the mutant conformation of the mutant p53 protein into a wild type conformation p53 protein, wherein the wild type conformation p53 protein possesses a biological activity of the wild type p53 protein.

2. The method of claim 1, further comprising: before the determining, obtaining a second sample, wherein the second sample comprises a second amount of the cell lysate;
i) contacting a first amount of the second sample with the antibody specific for the mutant conformation p53 protein in a third contacting area;
ii) contacting a second amount of the second sample with the antibody specific for the wild type conformation p53 protein in a fourth contacting area;
iii) quantifying an amount of the mutant conformation p53 protein in the third contacting area based on the contacting of the first amount of the second sample with the antibody specific for the mutant conformation p53 protein in the third contacting area;
iv) quantifying an amount of the wild type conformation p53 protein in the fourth contacting area based on the contacting of the second amount of the second sample with the antibody specific for the wild type conformation p53 protein in the fourth contacting area.

3. The method of claim 1, wherein the mutant p53 protein has a mutation at amino acid 220.

4. The method of claim 3, wherein the mutant p53 protein is Y220C mutant p53.

5. (canceled)

6. The method of claim 1, wherein the mutant p53 protein has a mutation at amino acid 273.

7. (canceled)

8. The method of claim 6, wherein the mutant p53 protein is R273H mutant p53

9. (canceled)

10. The method of claim 1, wherein the therapeutic candidate is a small molecule.

11. (canceled)

12. The method of claim 1, wherein the therapeutic candidate selectively binds the mutant p53 protein preferentially over the wild type p53 protein.

13. The method of claim 1, wherein the first contacting area is a first well with the antibody specific for the mutant conformation p53 protein immobilized to a surface within the first well, and the second contacting area is a second well with the antibody specific for the wild type conformation p53 protein immobilized to a surface within the second well.

14. The method of claim 1, wherein the quantifying the amount of the mutant conformation p53 protein comprises:
a) contacting the first amount of the sample with the antibody specific for the mutant conformation p53 protein, wherein the mutant conformation p53 and the antibody specific for the mutant conformation p53 form a first complex;
b) contacting the first complex with a detection antibody, wherein the first complex and the detection antibody form a second complex;
c) contacting the second complex with an enzyme, wherein the second complex and the enzyme form a third complex;
d) contacting the third complex with a substrate, wherein the contacting of the third complex with the substrate produces an oxidation product of the substrate; and
e) visualizing the oxidation product by spectrophotometry.

15. The method of claim 1, wherein the quantifying the amount of the wild type conformation p53 protein comprises:
a) contacting the first amount of the sample with the antibody specific for the wild type conformation p53 protein, wherein the wild type conformation p53 and the antibody specific for the wild type conformation p53 form a first complex;
b) contacting the first complex with a detection antibody, wherein the first complex and the detection antibody form a second complex;
c) contacting the second complex with an enzyme, wherein the second complex and the enzyme form a third complex;
d) contacting the third complex with a substrate, wherein the contacting of the third complex with the substrate produces an oxidation product of the substrate; and
e) visualizing the oxidation product by spectrophotometry.

16. The method of claim 1, wherein the quantifying the amount of the mutant conformation p53 protein comprises visualizing the amount of the mutant conformation p53 protein.

17. The method of claim 1, wherein the quantifying the amount of the wild type conformation p53 protein comprises visualizing the amount of the wild type p53 conformation protein.

18. The method of claim 16, wherein the visualizing comprises contacting the mutant conformation p53 protein with a detection antibody.

19. The method of claims 17, wherein the visualizing comprises contacting the wild type conformation p53 protein with a detection antibody.

20. (canceled)

21. The method of claim 18 or 19, wherein the visualizing further comprises contacting the detection antibody with an enzyme.

22-23. (canceled)

24. The method of claim 21, wherein the visualizing further comprises contacting the enzyme with a substrate.

25-29. (canceled)

30. The method of claim 1, wherein the cell lysate is obtained from a tumor.

31. The method of claim 30, wherein the tumor is a breast cancer tumor.

32. (canceled)

33. The method of claim 30, wherein the tumor is a lung cancer tumor.

34. (canceled)


SEQUENCE LISTING (36 sequences)

SEQ ID NO: 1 - protein, 9 aa, Artificial Sequence
  Description of Artificial Sequence Synthetic peptide
EVTFRHSVV

SEQ ID NO: 2 - protein, 19 aa, Artificial Sequence
  Description of Artificial Sequence Synthetic peptide
VWVHDSCHANLQNYRNYLP

SEQ ID NO: 3 - protein, 19 aa, Artificial Sequence
  Description of Artificial Sequence Synthetic peptide
FWTQSIKERKMLNEHDFER

SEQ ID NO: 4 - protein, 19 aa, Artificial Sequence
  Description of Artificial Sequence Synthetic peptide
EHDFEVRGDVVNGRNHQPK

SEQ ID NO: 5 - protein, 7 aa, Artificial Sequence
  Description of Artificial Sequence Synthetic peptide
LEVIYMI

SEQ ID NO: 6 - protein, 14 aa, Artificial Sequence
  Description of Artificial Sequence Synthetic peptide
LGIDEDEETETAPE

SEQ ID NO: 7 - protein, 20 aa, Artificial Sequence
  Description of Artificial Sequence Synthetic peptide
SPLQTPAAPGAAAGPALSPV

SEQ ID NO: 8 - protein, 12 aa, Artificial Sequence
  Description of Artificial Sequence Synthetic peptide
LTFEHYWAQLTS

SEQ ID NO: 9 - protein, 9 aa, Artificial Sequence
  Description of Artificial Sequence Synthetic peptide
LPNPPERHH

SEQ ID NO: 10 - protein, 16 aa, Artificial Sequence
  Description of Artificial Sequence Synthetic peptide
  N-term myristoyl
RRLIVRILKLPNPPER

SEQ ID NO: 11 - protein, 8 aa, Artificial Sequence
  Description of Artificial Sequence Synthetic peptide
LHSKTLVL

SEQ ID NO: 12 - protein, 11 aa, Artificial Sequence
  Description of Artificial Sequence Synthetic peptide
HHPWTHHQRWS

SEQ ID NO: 13 - protein, 12 aa, Artificial Sequence
  Description of Artificial Sequence Synthetic peptide
IRILMFLIGCGR

SEQ ID NO: 14 - protein, 21 aa, Artificial Sequence
  Description of Artificial Sequence Synthetic peptide
IRILMFLIGCGRRRRRRRRRR

SEQ ID NO: 15 - protein, 14 aa, Artificial Sequence
  Description of Artificial Sequence Synthetic peptide
DEDAKFRIRILMRR

SEQ ID NO: 16 - protein, 12 aa, Artificial Sequence
  Description of Artificial Sequence Synthetic peptide
LRCLLLLIGRVG

SEQ ID NO: 17 - protein, 20 aa, Artificial Sequence
  Description of Artificial Sequence Synthetic peptide
LRCLLLLIGRVGRRRRRRRR

SEQ ID NO: 18 - protein, 19 aa, Artificial Sequence
  Description of Artificial Sequence Synthetic peptide
YRRLLIGMMWRRRRRRRRR

SEQ ID NO: 19 - protein, 8 aa, Artificial Sequence
  Description of Artificial Sequence Synthetic peptide
YPTQGHLR

SEQ ID NO: 20 - protein, 7 aa, Artificial Sequence
  Description of Artificial Sequence Synthetic peptide
IRGRIIR

SEQ ID NO: 21 - protein, 15 aa, Artificial Sequence
  Description of Artificial Sequence Synthetic peptide
IRGRIIRRRRRRRRR

SEQ ID NO: 22 - protein, 14 aa, Artificial Sequence
  Description of Artificial Sequence Synthetic peptide
  N-term myristoyl
RRIRDPRILLLHFD

SEQ ID NO: 23 - protein, 12 aa, Artificial Sequence
  Description of Artificial Sequence Synthetic peptide
SFILFIRRGRLG

SEQ ID NO: 24 - protein, 21 aa, Artificial Sequence
  Description of Artificial Sequence Synthetic peptide
SFILFIRRGRLGRRRRRRRRR

SEQ ID NO: 25 - protein, 19 aa, Artificial Sequence
  Description of Artificial Sequence Synthetic peptide
RRRRRRRGLRGRRIFLIFS

SEQ ID NO: 26 - protein, 14 aa, Artificial Sequence
  Description of Artificial Sequence Synthetic peptide
  N-term myristoyl
RRKILFIRLMHNKH

SEQ ID NO: 27 - protein, 12 aa, Artificial Sequence
  Description of Artificial Sequence Synthetic peptide
HSSHHHPVHSWN

SEQ ID NO: 28 - protein, 13 aa, Artificial Sequence
  Description of Artificial Sequence Synthetic peptide
  N-term myristoyl
HSSHHHPTVQHRR

SEQ ID NO: 29 - protein, 7 aa, Artificial Sequence
  Description of Artificial Sequence Synthetic peptide
HANLHHT

SEQ ID NO: 30 - protein, 16 aa, Artificial Sequence
  Description of Artificial Sequence Synthetic peptide
  N-term myristoyl
RRKHNKHRPEPDSDER

SEQ ID NO: 31 - protein, 12 aa, Artificial Sequence
  Description of Artificial Sequence Synthetic peptide
WNHHHSTPHPAH

SEQ ID NO: 32 - protein, 18 aa, Artificial Sequence
  Description of Artificial Sequence Synthetic peptide
WNHHHSTPHPAHRRRRRR

SEQ ID NO: 33 - protein, 9 aa, Artificial Sequence
  Description of Artificial Sequence Synthetic peptide
  N-term myristoyl
RRHSTPHPD

SEQ ID NO: 34 - protein, 7 aa, Artificial Sequence
  Description of Artificial Sequence Synthetic peptide
FPGHTIH

SEQ ID NO: 35 - protein, 16 aa, Artificial Sequence
  Description of Artificial Sequence Synthetic peptide
  N-term myristoyl
PRVLPSPHTIHPSQYP

SEQ ID NO: 36 - protein, 219 aa, Artificial Sequence
  Description of Artificial Sequence Synthetic polypeptide
SSSVPSQKTYQGSYGFRLGFLHSGTAKSVTCTYSPALNKMFCQLAKTCPVQLWVDSTPPPGTRVRAMAIYKQSQHMTEVVRRCPHHERCSDSDGLAPPQHLIRVEGNLRVEYLDDRNTFRHSVVVPCEPPEVGSDCTTIHYNYMCNSSCMGGMNRRPILTIITLEDSSGNLLGRNSFEVHVCACPGRDRRTEEENLRKKGEPHHELPPGSTKRALSNNT